(19) United States
(12) Patent Application Publication
(10) Pub. No.: US 2024/0341566 A1
(43) Pub. Date: Oct. 17, 2024

(54) SYSTEM AND METHOD FOR LOCATING AND IDENTIFYING MEDICAL TARGET OBJECTS

(71) Applicant: KARL STORZ SE & Co. KG, Tuttlingen (DE)

(72) Inventor: Klaus Brüderle, Tuttlingen (DE)

(73) Assignee: KARL STORZ SE & Co. KG, Tuttlingen (DE)

(21) Appl. No.: 18/635,679

(22) Filed: Apr. 15, 2024

(30) Foreign Application Priority Data
Apr. 17, 2023 (DE) 10 2023 109 620.6

(51) Int. Cl. A61B 1/00

(52) U.S. Cl. CPC A61B 1/00004; A61B 1/00158

(57) ABSTRACT

A system for locating and identifying a plurality of medical target objects, the system comprising the plurality of medical target objects and a measuring device for locating and identifying the plurality of medical target objects, with the plurality of medical target objects creating different magnetic fields, the position, direction and source of which can be determined. A method for locating and identifying a plurality of medical target objects is also disclosed.

### CROSS-REFERENCE TO RELATED APPLICATIONS

This application claims priority of German Patent Application No. 10 2023 109 620.6 filed on Apr. 17, 2023, the contents of which are incorporated herein.

### TECHNICAL FIELD

The present invention relates to a system for locating and identifying medical target objects and to a method for locating and identifying medical target objects.

### BACKGROUND

In the field of medical engineering, endoscopes are usually used to pictorially capture interior structures. This allows diagnoses to be made or practical surgery to be performed. This is mainly based on the circumstances being assessed optically, i.e. in the visible range, and the actions being selected and carried out on the basis of this assessment.

These optical records relate to the superficial situation. They must be assessed in relation to the patient; hence, a spatial reference and an identification of direction are important. While the effective application occurs within the body of the patient, the operation, in particular the positioning, is implemented from the outside. Thus, the signals must overcome the body barrier, i.e. be guided from the interior to the exterior. In this context, preference is given to forms of transmission that are able to penetrate skin or bone in particular, i.e. require no additional port.

The spatial acquisition of the direction and the position of a target object requires an appropriate signal generator which is connected to the target object or the part of the target object in a spatially defined manner, especially in a fixed arrangement. If the target object is situated within the body, the signal from the signal generator is detected outside of the body and further processed appropriately and used for determining the location or navigating, for example.

Approaches for overcoming the body barrier have already been disclosed. For example, US 2014/0051985 A1 discloses a target finding system which identifies a surgical target, e.g. a renal calculus, by virtue of arranging a transmitter, e.g. a magnetic source, behind or next to the surgical target and using a circuit to identify an axis to the transmitter and hence define an axis or path to the surgical target. An array of sensors arranged in an equidistant, coplanar arrangement in each case acquires a signal specifying the distance from the emitter. A magnetic resistance sensor, which creates a variable resistance, reacts to the distance from a solenoid which emits a magnetic field. An identical signal from each of the coplanar sensors indicates the positioning on an axis that runs through a point in the middle of the sensors and orthogonal to the plane.

### SUMMARY

It is an object of the present invention to highlight an improved system and an improved method for locating and identifying medical target objects.

According to a first aspect, the object is achieved by a medical system for locating and identifying a plurality of medical target objects, the system comprising the plurality of medical target objects and a measuring device for locating and identifying the plurality of target objects, with the plurality of medical target objects comprising a first medical target object and a second medical target object, the first medical target object being designed to create a first magnetic field with a first time profile, the second medical target object being designed to create a second magnetic field with a second time profile, which differs from the first time profile, and the measuring device being designed to use a magnetic field sensor to detect magnetic field lines of the first magnetic field and second magnetic field with regards to their direction, their strength and their time profile, and to use a processing device to ascertain a position and orientation of the respective target object from the detected direction and strength, to assign the ascertained position and orientation to one of the medical target objects from the temporal occurrence and to output the position and orientation for the assigned medical target object.

An advantage of this solution is that it is possible to set up more than only one communications channel. Specifically, it is possible to acquire the information, i.e. position and orientation, of a plurality of medical target objects and respectively assign this information unambiguously to one of the medical target objects. As a consequence, it is now possible to track and monitor the work with a plurality of target objects.

The aforementioned temporal occurrence is detected here by way of the profile of one of the magnetic fields. To this end, it is preferable that a plurality of changes are acquired by way of the magnetic field sensor so that the detected changes can be used to deduce the target object causing these changes. To this end, each target object has, in particular, its own signature, clocking, frequency, intervals, clock sequence or amplitude variance. Given knowledge of the expected shape of the used signal, a person skilled in the art knows the number of samples required to distinguish one used signal from another used signal, and so this aspect will not be discussed in detail here.

Moreover, the proposed solution offers further advantages. Firstly, there is a good signal-to-noise ratio. The general parameters of the application are such that the expected potential interference signal is higher than the used signal. However, the proposed solution allows good identification of the used signal despite the disadvantageous parameters. Moreover, the sought-after used signal can be isolated from other ambient signals, so that these overlaid influences are kept small. Moreover, magnetic signals pass through body barriers such as skin and bones without loss of information and are not influenced or disturbed or only influenced or disturbed to a negligible extent as a result.

Moreover, identification is implemented in “stateless” fashion and at an atomic level. Thus, no knowledge of a previous position and/or orientation profile is required or no consideration needs to be given thereto. As a result, the proposed solution differs from other methods which acquire the position by adding the movement paths of the target object. In this case, the displacements are acquired and processed continuously by way of acceleration sensors. This requires knowledge of the gap-free sequence of the movement paths.

The spatial characteristic of the magnetic field of the respective target object is known. Knowing this characteristic then allows the position in the magnetic field at which the magnetic field sensor is located to be deduced from the measured direction and strength. From this, it is possible in turn to ascertain the location of the target object, in particular a defined reference point of the target object.

The magnetic field of the respective target object is able to be switched on and off in some configurations; in other preferred configurations, it is created by a solenoid and can be controlled by controlling the current flow through the solenoid. In such a configuration, the solenoid is controllable by way of electrical pulses.

The used signal is a magnetic signal; more accurately, the field lines, in particular the field line envelope curve of the magnetic field, are the used signal. Thus, this used signal has an elliptic shape, i.e. is a spatial signal determined with regards to strength, direction and position. The magnetic field sensor uses the magnetic part to detect the magnetic spatial signal (X, Y, Z). In this context, the spatial signal can be imagined to be a three-dimensional body which represents this elliptic shape, i.e. the bodily shape of the signal. It is further possible to imagine that this elliptic shape is formed from many small segments, each of which can be described as a vector. The position and orientation of the respective target object can be ascertained by virtue of identifying these vectors or directional vectors.

This completely achieves the object.

In a preferred configuration, the magnetic fields are each created by a pulse-type trigger.

The magnetic field sensor reacts to all magnetic influences, such as the Earth's magnetic field, running motors, steel girders, etc. However, these are static or at least defined dynamic signal shapes (50 Hz or higher). Triggering by pulses, in particular triggering a solenoid, can be used here in such a way that these ambient signals can be separated from the used signal by means of an evaluation filtering.

Moreover, this pulse controllability, i.e. switching on and off in particular, can also be designed such that the resultant used signals can be overlaid. Then, a source identification is present in the control clock. This source identification is an impressed piece of meta information. This achieves the desired duality of, firstly, a piece of spatial signal information for direction and position and, secondly, a piece of meta signal information for channel or source identification and additionally for the time profile. Thus, this is a digital information transfer from the source, i.e. the target object, to the receiver, i.e. the measuring device. Over time, the control clock can also be modified in terms of its frequency and/or amplitude such that a certain source can alternatively or additionally also be identified on the basis of a change over time of the magnetic field with regards to frequency and/or amplitude.

In a preferred configuration, the magnetic fields are each created by a wave-shaped trigger, in particular by a sinusoidal trigger.

On the one hand, there is the option here of identifying a source on the basis of its employed wave shape, e.g. sinusoidal shape, rectangular shape, triangular shape, sawtooth shape, etc. To this end, the time profile of the spatial signal is measured at one location in the room, in order to be able to deduce the source from the measured time profile. In an alternative or in addition, the frequency and/or the amplitude of the wave-shaped trigger can also be chosen and determined at a later stage from the time profile of the spatial signal in order to be able to deduce the source. In an alternative or in addition, the frequency and/or the amplitude of the wave-shaped trigger can also be modified over time. Then, the change in frequency and/or amplitude is ascertained from the time profile of the spatial signal in order to be able to deduce the source. Thus, it is possible to differentiate between different signal sources in many different ways, wherein the features of shape, frequency, amplitude, change in shape, change in frequency and change in amplitude can be used in any combination or on their own for the purpose of identifying a source.

In a further preferred configuration, the first target object creates the first magnetic field periodically at a first frequency, the second target object creates the second magnetic field periodically at a second frequency and the measuring device comprises a variable bandpass filter, the mid-frequency of which can be selectively set to the first frequency or the second frequency.

In principle, a temporal separation with regards to when the first target object and the second target object create the respective magnetic field can ensure that the magnetic fields can be easily distinguished. However, this configuration allows the magnetic fields to be distinguished even when they overlap in time. The bandpass filter in each case allows dominant acquisition of a signal of a desired magnetic field.

In a further preferred configuration, each magnetic field is created by a respective current which is a rectangular signal or a sawtooth signal or a triangular signal or particularly preferably a sinusoidal signal.

This configuration allows good separation between the magnetic fields.

In a further preferred configuration, the magnetic field sensor is configured as a component of a tablet, in particular as part of a gyroscope sensor of the tablet.

This allows a cost-effective solution to be obtained, and the latter moreover is incorporated seamlessly in the already present use of a tablet, for example for viewing and inputting data.

In a further preferred configuration, the first target object is arranged at a spatially fixed location in the room, in particular on an operating table.

This allows a spatial reference of the target object to be established with respect to the work surroundings. To this end, the target object may be installed outside of the body, in particular in an operating table, and preferably in a foot of the operating table. In that case, the material is V2A or stainless steel in particular. A target object that is fixedly positioned in this way is preferably used for differential correction, as known from differential GPS, for example.

In a further preferred configuration, the distance between the measuring device and each of the target objects is no more than 3 m, preferably between 0.5 m and 2.5 m, and particularly preferably between 1 m and 2 m.

On account of the short distance, i.e. the small effective range, the required signal intensity is low. For example, it is orders of magnitude smaller than in an MRI device or magnetic resonance imaging scanner.

In a further preferred configuration, the measuring device comprises a plurality of magnetic field sensors which are spaced apart from one another and based on the same functional principle, and the measuring device is designed to check positions and orientations of the respective target object as ascertained by the individual magnetic field sensors against one another with regards to plausibility and/or to improve the accuracy of the position and orientation by averaging.

In this configuration, the signal is received and evaluated at a plurality of different positions. That is to say, two or more sensors evaluate the spatial ellipse of the magnetic field of a medical target object, and the result should at least approximately yield the same position data for the associated medical target object. This means that the acquisition is verified or checked for plausibility at the current time, and hence there is greater reliability. In addition or in an alternative, the accuracy can be improved by averaging the measurement values. As a result of a plurality of measurements being performed at different positions, this can also be referred to as a two-factor evaluation, which increases the accuracy.

In a further preferred configuration, the second target object is fixedly arranged on an endoscope.

As a result, the frequent use of an endoscope as mentioned at the outset can advantageously be improved.

According to a second aspect, the object is achieved by a method for locating and identifying a plurality of medical target objects, the method including the following steps: creating a first magnetic field with a first time profile by way of a first medical target object; creating a second magnetic field with a second time profile, which differs from the first time profile, by way of a second medical target object; detecting, by means of a magnetic field sensor, magnetic field lines of the first magnetic field and second magnetic field with regards to their direction, their strength and their time profile; ascertaining, by means of a processing device, a position and orientation of the respective medical target object from the direction and the strength; assigning, by means of the processing device, the ascertained position and orientation to one of the medical target objects on the basis of the time profile; and outputting the position and orientation for the assigned medical target object.

It will be appreciated that the features mentioned above and the features yet to be explained below are applicable not only in the combination specified in each case but also in other combinations or on their own, without departing from the scope of the present invention.

### DETAILED DESCRIPTION OF DRAWINGS

An exemplary embodiment of the invention is depicted in the drawings and described in detail in the following description. In the drawings:

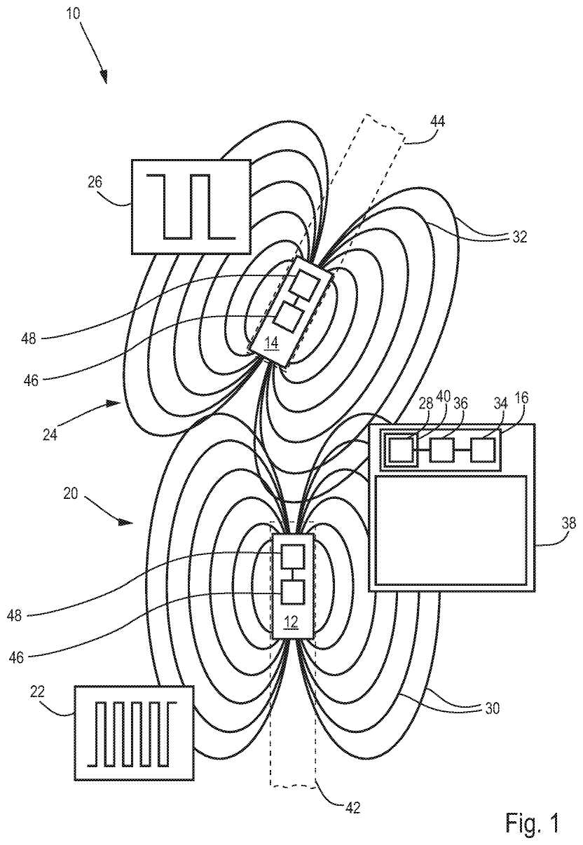

FIG. 1 shows an embodiment of a medical system for locating and identifying a plurality of target objects;

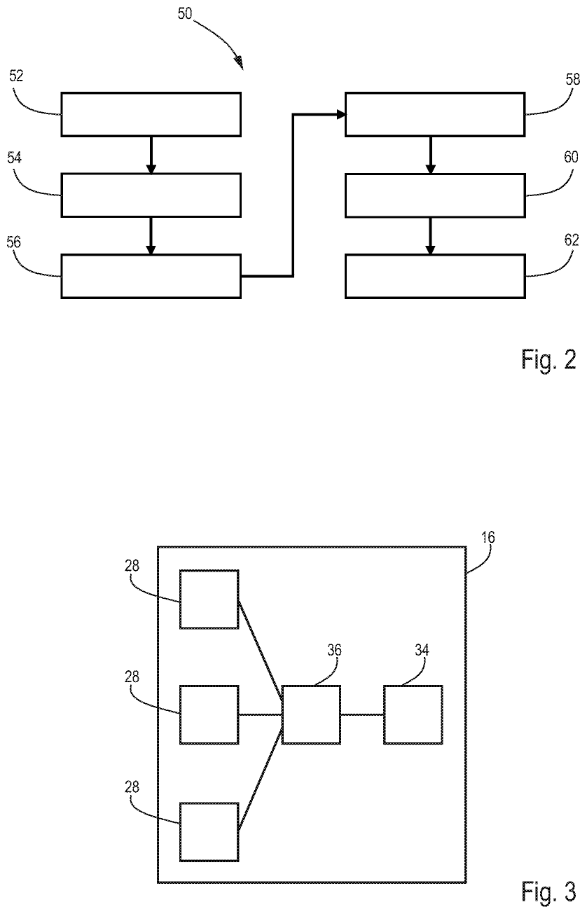

FIG. 2 shows an embodiment of a method for locating and identifying a plurality of medical target objects; and

FIG. 3 shows an embodiment of a measuring device having a plurality of magnetic field sensors.

### DETAILED DESCRIPTION OF PREFERRED EMBODIMENTS

FIG. 1 shows an embodiment of a medical system 10 for locating and identifying a plurality of medical target objects 12, 14. The system 10 includes the plurality of target objects 12, 14 and a measuring device 16 for locating and identifying the plurality of target objects 12, 14.

Specifically, the plurality of target objects 12, 14 comprise a first target object 12 and a second target object 14. In this case, the first target object 12 is designed to create a first magnetic field 20 with a first time profile 22, and the second target object 14 is designed to create a second magnetic field 24 with a second time profile 26, which differs from the first time profile 22.

The measuring device 16 is designed to detect, by means of a magnetic field sensor 28 of the measuring device 16, magnetic field lines 30, 32 of the first magnetic field 20 and second magnetic field 24 with regards to their direction, their strength and their time profile.

Then, a processing device 34 of the measuring device 16 is used to ascertain a position and orientation of the respective target object 12, 14 from the detected direction and strength. This is possible since the expected shape of the magnetic field 20, 24 of a target object 12, 14 is known. Knowledge about the shape of the magnetic field 20, 24 can be used to deduce the location in this shape at which the magnetic field sensor 28 is located, with the result that this in turn allows the origin of the magnetic field 20, 24, i.e. the target object 12, 14 or a reference point of the target object 12, 14, to be deduced relative to the magnetic field sensor 28.

The ascertained position and orientation is assigned to one of the target objects 12, 14 by the processing device 34 on the basis of the temporal occurrence of the magnetic field 20, 24. This is possible since the target objects 12, 14 create their respective magnetic field with different time profiles 22, 26. For example, if the first magnetic field 20 is switched on and off quickly and the second magnetic field 24 is switched on and off slowly, then the magnetic fields can be distinguished on account of the time profile 22, 26 of the resultant magnetic fields 20, 24. To this end, the magnetic fields 20, 24 can each be created by a solenoid 46 triggered in pulse-type fashion by a control unit 48.

Finally, the processing unit 34 is designed to output the position and orientation for the assigned target object 12, 14. This information can be used in particular to display the respective target object 12, 14 virtually, for example on an electronic visual display or on a VR headset. As a result, the user can intuitively grasp the spatial situation even though they have no line of sight to the respective target object 12, 14.

In the embodiment shown here, the first medical target object 12 creates the first magnetic field 20 periodically at a first frequency and the second medical target object 14 creates the second magnetic field 24 periodically at a second frequency. The measuring device 16 comprises a variable bandpass filter 16, the mid-frequency of which can be selectively set to the first frequency or the second frequency. This allows separation of the acquisition of the two magnetic fields 20, 24, even if these superimpose intermittently.

In this case, the magnetic field sensor 28 is designed as a component of a tablet 38, in particular as part of a gyroscope sensor 40 of the tablet 38. Moreover, the first target object 12 is arranged in this case at a spatially fixed location in the room, especially on an operating table 42 indicated here using dashed lines. The second target object 12 is fixedly arranged on an endoscope 44, the latter likewise indicated using dashed lines.

FIG. 2 shows an embodiment of a medical method 50 for locating and identifying medical target objects 12, 14. The method 50 starts with the creation 52 of a first magnetic field 20 with a first time profile 22 by way of a first medical target object 12. There is also the creation 54 of a second magnetic field 24 with a second time profile 26, which differs from the first time profile 22, by way of a second medical target object 14.

This is followed by the step of detecting 56, by means of a magnetic field sensor 28, magnetic field lines 30, 32 of the first magnetic field 20 and second magnetic field 24 with regards to their direction, their strength and their time profile 22, 26. In a further step, there is an ascertainment 58, by means of a processing device 34, of a position and orientation of the respective target object 12, 14 from the direction and the strength.

In the next step, there is an assignment 60, by means of the processing device 34, of the ascertained position and orientation to one of the target objects 12, 14 on the basis of the time profile. Finally, there is the output 62 of the position and orientation for the assigned target object 12, 14.

FIG. 3 shows an embodiment of a measuring device 16 having a plurality of magnetic field sensors 28, which are spaced apart from one another and based on the same functional principle. In this embodiment, the measuring device 16 is designed to check positions and orientations of the respective target object 12, 14 as ascertained by the individual magnetic field sensors 42 against one another with regards to plausibility and/or to improve the accuracy of the position and orientation by averaging.

1. A medical system comprising:
a plurality of medical target objects including a first medical target object and a second medical target object, wherein the first medical target object is configured to create a first magnetic field with a first time profile, and the second medical target object is configured to create a second magnetic field with a second time profile, which differs from the first time profile, and
a measuring device configured to use a magnetic field sensor to detect a magnetic field line of the first magnetic field and second magnetic field and detect a direction, a strength and a time profile, and to use a processing device to ascertain a position and orientation of the respective target object from the detected direction and strength, to assign the ascertained position and orientation to one of the target objects from the temporal occurrence and to output the position and orientation for the assigned medical target object.

2. The medical system as set forth in claim 1, wherein the magnetic fields are each created by a pulse-type trigger.

3. The medical system set forth in claim 1, wherein the magnetic fields are each created by a wave-shaped trigger, in particular by a sinusoidal trigger.

4. The medical system set forth in claim 1, wherein the first medical target object creates the first magnetic field periodically at a first frequency, the second medical target object creates the second magnetic field periodically at a second frequency and the measuring device comprises a variable bandpass filter, the mid-frequency of which can be selectively set to the first frequency or the second frequency.

5. The medical system set forth in claim 4, wherein each magnetic field is created by a respective current which is a rectangular signal or a sawtooth signal or a triangular signal or particularly preferably a sinusoidal signal.

6. The medical system set forth in claim 1, wherein the magnetic field sensor is configured as a component of a tablet, in particular as part of a gyroscope sensor of the tablet.

7. The medical system set forth in claim 1, wherein the first medical target object is arranged at a spatially fixed location in the room, in particular on an operating table.

8. The medical system as set forth in claim 1, wherein the distance between the measuring device and each of the medical target objects is no more than 3 m, preferably between 0.5 m and 2.5 m, and particularly preferably between 1 m and 2 m.

9. The medical system as set forth in claim 1, wherein the measuring device includes a plurality of magnetic field sensors which are spaced apart from one another and based on the same functional principle, and the measuring device is designed to check positions and orientations of the respective medical target object as ascertained by the individual magnetic field sensors against one another with regards to plausibility and/or to improve the accuracy of the position and orientation by averaging.

10. The medical system as set forth in claim 1, wherein the second target object is fixedly arranged on an endoscope.

11. A method for locating and identifying a plurality of medical target objects, the method including the following steps:
creating a first magnetic field with a first time profile by way of a first medical target object;
creating a second magnetic field with a second time profile, which differs from the first time profile, by way of a second medical target object;
detecting, by means of a magnetic field sensor, magnetic field lines of the first magnetic field and second magnetic field with regards to their direction, their strength and their time profile;
ascertaining, by means of a processing device, a position and orientation of the respective medical target object from the direction and the strength;
assigning, by means of the processing device, the ascertained position and orientation to one of the medical target objects on the basis of the time profile; and
outputting the position and orientation for the assigned medical target object.
